(19) United States
(12) Patent Application Publication
(10) Pub. No.: US 2005/0153382 A1
(43) Pub. Date: Jul. 14, 2005

(54) Biochip kit comprising biochip based on antigen-antibody reactions, and its usage

(75) Inventors: Fanglin Zou, Chengdu City (CN); Chunsheng Chen, Chengdu City (CN); Jianxia Wang, Meishan City (CN)

Correspondence Address: BIRCH STEWART KOLASCH & BIRCH, PO BOX 747, FALLS CHURCH, VA, 22040-0747, US

(73) Assignees: Chengdu Kuachang Science and Technology Co., Ltd., Chengdu City (CN); Human Plasma Product Services Asia Ltd., Hong Kong (CN)

(21) Appl. No.: 11/000,944

(22) Filed: Dec. 2, 2004

Related U.S. Application Data
(63) Continuation of application No. PCT/CN03/00026, filed on Jan. 14, 2003.

(30) Foreign Application Priority Data
Jun. 6, 2002 (CN) 02113834.6

(51) Int. Cl. G01N 33/53; G01N 33/537; G01N 33/543; C12M 1/34

(52) U.S. Cl. 435/7.93; 435/287.2

(57) ABSTRACT

This invention concerns a biochip kit, comprising at least one biochip with at least one reactor, in which probe antibody and probe antigen are immobilized non-randomly in the form of probes array or/and probes pattern. This invention also concerns a method for testing a biological sample by using the kit. This kit can be used to analyze different target molecules corresponding to said probe antibody and probe antigen. By using this kit, the reactor number and the detection time needed for the analysis are reduced while the detection comparability is increased. So this kit has the advantage of being economical, timesaving and more efficient.

This application is a Continuation of copending PCT International Application No. PCT/CN03/00026 filed on Jan. 14, 2003, which designated the United States, and on which priority is claimed under 35 U.S.C. § 120. This application also claims priority under 35 U.S.C. § 119(a) on Patent Application No(s). 02113834.6 filed in China on Jun. 6, 2002. The entire contents of each of the above documents is hereby incorporated by reference.

### TECHNICAL FIELD

This invention pertains generally to biochip kit for analyzing biological sample qualitatively and/or quantitatively. In particular, this invention involves a biochip kit used for combined assay of different target molecules, e.g. target antibody and target antigen in one sample. The kit comprises at least one biochip with at least one reactor, in which at least probe antibody and probe antigen are immobilized non-randomly in the form of probes array or/and probes pattern, to capture different target molecules respectively. If marking is needed, one marker combination of different markers can bind the target molecules captured. This marker combination comprises different ligands corresponding to different probes, thereby the reaction results can be analyzed.

This invention also involves a method using this biochip kit.

### INVENTION BACKGROUND

At present, biochip kit based on antibody-antigen reactivity comprises biochip with either probe antigens or probe antibodies. The biochip with probe antigens comprises one or more reactors, in which multiple antigens are immobilized as the probes in a non-random form (e.g. in the address-targeted form). And the biochip with probe antibodies comprises one or more reactors, in which multiple antibodies are immobilized as the probes in a non-random form.

The present biochip kit often comprises marking system, in which marker is either labeled antigen or labeled antibody or labeled anti-antibody. The labeled antigen or labeled anti-antibody reacts with antibody captured, and the labeled antibody reacts with antigen captured. So, the present biochip kit is used for detecting either multiple antibodies or multiple antigens in a sample, corresponding to the multiple antigens or multiple antibodies used as the probes, and labeled antigen or antibody or anti-antibody used as marker.

The reaction between the reagents (probe molecules, target molecules, markers, etc.) in a reactor may be performed in different reaction patterns, such as indirect detection pattern, capturing pattern, competitive inhibition pattern, bi-antibody sandwich detection pattern and bi-antigen sandwich detection pattern, etc. So the detection methods corresponding to the reaction patterns are indirect detection method, capturing method, competitive inhibition method, bi-antibody sandwich detection method and bi-antigen sandwich detection method etc. A present biochip can be usually used with the different detection methods. However, a present kit comprising the biochip and the marker can be only used with a defined detection method. For example, for a biochip with antigens immobilized, which captures respectively the target antibodies in a sample, the bi-antigen sandwich detection method should be used if the markers are labeled specific antigens reactive with the target antibodies captured respectively; the indirect detection method should be used if the marker is labeled anti-antibody reactive with the target antibodies captured. While for a biochip with antibodies immobilized which capture the target antigens in a sample, the bi-antibody sandwich detection method should be used if the markers are labeled specific antibodies reactive with the target antigens captured respectively.

The biochip kit based on antibody-antigen reactivity has extensive application, especially in the clinical immunology diagnosis. However, the present biochip kits can be only used for detecting either target antigens or target antibodies, which limits its efficiency or/and its application.

### INVENTION CONTENT

This invention provides a biochip kit comprising at least one biochip with at least one reactor, in which at least probe antibody and probe antigen are immobilized non-randomly in the form of probes array or/and probes pattern.

The kit comprises also markers consisting of ligands corresponding to said probe antibody and probe antigen and label reagent bound to the ligands. Said ligand presents specific antibody-antigen reactivity, or antigen affinity or/and antibody affinity.

In the kit, said markers are one of the following marker combinations:
A. the combination of labeled specific antibody and labeled specific antigen;
B. the combination of labeled anti-antibody with different structure-specificity ;
C. the combination of labeled anti-antibody with species-specificity and labeled specific antibody and/or labeled specific antigen;
D. the combination of labeled anti-antibody with structure-specificity and labeled specific antibody and/or labeled specific antigen;
E. any combination derived from the combinations mentioned above.

In the kit, said probe antibody includes HBsAb (antibody against Hepatitis B surface antigen) and said probe antigen includes human immuno-deficient virus (HIV) antigen, hepatitis C virus (HCV) antigen and syphilis antigen; or said probe antibody includes also hepatitis B e antibody (HBeAb) and said probe antigen includes hepatitis B core antigen (HBcAg), hepatitis B surface antigen (HbsAg) and hepatitis B e antigen (HBeAg); or said probe antigen includes hepatitis C virus antigen (HCV-Ag) and hepatitis G virus antigen (HGV-Ag).

In the kit, said probe antibody includes antibody against anti-HIV p24 antigen, and said probe antigen includes HIV gp160, HIV gp41and HIV gp36 epitopes.

In the kit, said probe antigen includes Epstein-Barr virus (EBV) antigen (EBV- Ag). In the kit, said probe antibody includes: antibody against human chorionic gonadotrophin(HCG), anti-tumor marker antigen 50 (CA50) antibody, anti-saccharide antigen 242 (CA242) antibody, anti-mammary cancer specific antigen (CA153) antibody, anti-carcinoembryonic antigen (CEA) antibody, and anti-oophoroma specific antigen (CA125) antibody.

This invention provides also a method of detecting biological sample. The method comprises:
(1) Subjecting the biological sample to the reactor in the biochip of any one of claim 1-10, and making them react;
(2) Optionally, subjecting marking reagents to said reactor in the form of individual substance, partial mixture or complete mixture, and
(3) Analyzing results in the reactor after the reaction.

### DETAILED DESCRIPTION OF THE INVENTION

Scientists have long recognized the following antigen-antibody reactivities: the specific reactivity between an antigen and its pair antibody; the species-specific reactivity based on different reactivities of an antibody with different anti-antibodies prepared from different species of animals; and the structure-specific reactivity based on different reactivities of a type or subtype of immunoglobulin with different anti-antibodies prepared by using different types or subtypes of immunoglobulin or their characteristic segments. However, this scientific knowledge has not been applied to the combination assay of different target molecules in one biochip reactor (e.g. antigen and antibody combination assay), and it has been thought that there is undesired cross-reaction among the reagents (probe molecules, target molecules, markers, etc.) in reactor. However, it is surprising that no cross-reaction is observed by applying this knowledge in our biochip study on the cross-reaction (Table 1).

TABLE 1
The experiment results on the cross-reaction among
antigen/antibody/Anti-antibodies

| reactor | | ligand in | cross-reaction |
|---|---|---|---|
| No. | probes | fluorescent marker | result* |
| 1 | rabbit antibody | Mouse mAb against | − |
| | against HBsAg | HBsAg | |
| | Mouse mAb | | − |
| | against HBsAg | | |
| 2 | Rabbit antibody | goat anti-Ab against | − |
| | against HBsAg | human antibody | |
| | Mouse mAb | | − |
| | against HBsAg | | |

*− means negative and + means positive

So, in an embodiment, the biochip kit of this invention is characterized by the biochip with at least one reactor, in which at least probe antibody and probe antigen are immobilized non-randomly in the form of probes array or/and probes pattern.

In this invention, the “biochip kit” or “kit” refers to an indispensable consumable, comprising one or more biochip, in quantitative and/or qualitative biochip analysis; the “biochip” or “probe-plate” refers to a chief component of the kit, comprising one or more reactors for the quantitative and/or qualitative biochip analysis; the “biochip reactor” or “reactor”, a kernel of the biochip, refers to a boundary-defined continuous surface or container, where the probes are immobilized directly or indirectly and can react with target molecules of a subjected sample with identifiable reaction results, e.g. a probe plate of a mono-reactor chip or a reaction well of a multi-reactor biochip.

So, the biochip kit in this invention is different from the present routine test kit and the present rapid test kit. In the present routine test kit, such as Enzyme-linked immunosorbent assay (ELISA) kit, biotin-avindin assay kit, radio-immunoassay (RIA) kit, immune-fluorescence kit, chemiluminescence kit, electrochemiluminescence kit, etc., the probe is immobilized randomly in a reactor. In the present rapid test kit, either probe antigen or probe antibody is immobilized non-randomly on a reactor (e.g. a test strip based on immune-affinity chromatography) in the form of probe line but not in the form of probe micro-array, which limits the probe number. The biochip reactor in this invention is different also from device combining several reactors, e.g. a device combining several test strips (Chinese patent application number 00226807.8), in which either probe antigen or probe antibody is immobilized as probe on each of the strips.

In this invention, the “probe” refers to indispensable active substance immobilized in the reactor for capturing the target molecule in a sample. In the biochip kit of this invention, the probe immobilized in the reactor comprises at least the probe antibody and probe antigen. Other active substance beside the probe antibody and probe antigen can be used also as the probe in this invention, if its presence and application do not introduce the problem of cross-reaction.

In this invention, the “antigen (Ag)” refers to an important substance in immunoassay, which can directly or indirectly stimulate immune response to produce antibodies and/or primed lymphocytes, and develop immune response at the same time by binding specifically to the immunity-responding products (i.e. antibody and/or primed lymphocyte). The antigen contains: complete antigen, haptin and other substance with immune reactivity. Some examples of the antigen are the followings: A. the component of plant, animal and microorganism with immunogenicity and immune reactivity (e.g. protein, peptides, lipid, polysaccharide, saccharide and other organic ingredients as well as combinations thereof); B. the metabolism products of plant, animal and microorganism; C. artificial antigen; D. synthetic antigen; E. genetic engineering recombinant antigen; F. medicine or ingredient of medicine; G. antibody used for preparing anti-antibody, or anti-antibody for preparing anti-anti-antibody.

In this invention, the “antibody (Ab)” contains: natural antibody, specific antibody, polyclonal antibody, monoclonal antibody (mAb), genetic engineering antibody and antibody fragment; the “anti-antibody (anti-Ab)” means an antibody prepared from animal immunization by using another antibody or another anti-antibody as an antigen. The anti-antibody includes: antibody against an antibody, antibody against an anti-antibody, or antibody against an anti-anti-antibody, etc. In this invention, the “immunoglobulin (IG)” contains: different types of immunoglobulins (IgA, IgG, IgM, IgE, IgD), and different subtypes of immunoglobulins (IgG1, IgG2, IgG3, IgG4).

In this invention, the “probe antibody and antigen” refers to antibody and antigen immobilized on the biochip reactor and used as probe. The probe antibody or antigen can be natural or synthetic material, containing polypeptide, protein and all other substance with the properties of antibody or antigen. An example of the probe antibody and antigen is a probe combination comprising 1 antibody (antibody against HBsAg) and 4 antigens (HCV antigen, HIV₁₊₂ antigen, HTLV antigen and syphilis antigen) immobilized in a reactor of the biochip, and used to capture 1 antigen (HbsAg) and 4 antibodies (HCV antibody, HIV₁₊₂ antibody, HTLV antibody and syphilis antibody). More examples will be presented in the following part “EXAMPLES”.

In this invention, the probe antibody and probe antigen immobilized in the reactor of subject biochip are either paired or non-paired. In this invention, the “paired antigen and antibody” refers to antigen and antibody which can interact each other, e.g. HBsAg and HBsAb used as probe antigen and probe antibody in Example 6; the “non-paired antigen and antibody” refers to antigen and antibody which cannot interact each other, e.g. the combinations of probe antigen and probe antibody used respectively in Examples 1-5, 7 and 8.

In this invention, the “non-random immobilization” of the probe antibody and antigen means an immobilization by which the different probe antibody and antigen immobilized can be identified geometrically; the “probes array” means a probe arrangement in the form of array; the “probes pattern” means a probes distribution, in which the probes are immobilized non-randomly in the form of recognizable pattern. As all known, the probe points in a probes array are separated from each other, which are address-traceable. An example of the probes array is a probes array composed of M types of antigens (M≧1) and N types of antibodies (N≧1) immobilized in the form of (M+N)×L array (L is the spot number of each type of the probes). More examples will be presented in the following part “EXAMPLES”. In the EXAMPLES, no cross-reaction has been observed, showing that no cross-reaction among the probe antibody and antigen has been produced in the reactor of the biochip of this invention.

In this invention, the probes are immobilized directly or indirectly in the reactor. An example for the indirect immobilization is: different kinds of probes are immobilized on the activated particles separately, and then the activated particles bound to the probes are immobilized on the substrate of the biochip reactor. Whether the probes are immobilized directly or indirectly will not substantially affect the detection results of this biochip for different target molecules combinations described in this invention. In this invention, the “substrate” refers to a solid support for immobilizing the probes. Material for making the substrate contains various organic, inorganic, transparent or opaque, water-absorbable or water-unabsorbable, hard or soft materials, such as one or several kinds of following materials: glass, plastic, rubber, metal, cellulose membrane, slice, plate, etc. In production of the invented kit, various probes (probe antigen and probe antibody) can be immobilized on the substrate simultaneously or successively.

The biochip kit of this invention is characterized also by its marking system. The invented biochip kit comprises markers consisting of ligands, corresponding to said probe antibody and probe antigen, and label reagent bound on the ligands. These ligands present respectively specific antibody-antigen reactivity, or antigen affinity or/and antibody affinity.

In this invention, the “substance to be marked” means a molecule to be made react with the marker, e.g. a target antigen, a target antibody, or an intermediate connecting a target antigen or a target antibody; the “marking system”, an important component of the kit, refers to a mass of materials used to ‘mark’ the results of the reaction between the probes and the target molecules in a quantitative and/or qualitative assay. The marking system includes the markers. It includes also, in some cases, intermediates, label amplifying agent and so on. Some examples of the marking system with label amplifying agent are: the colloidal gold marking system with amplifying silver-salt, the marking system with amplifying biotin and/or avidin and so on.

The marking system in the subject biochip kit adopts the following labeling methods or their combinations to mark the specific reactions: gold (silver) labeling method, fluorescence labeling method, chemiluminescence labeling method, electrochemiluminescence labeling method, radioactive labeling method or magnetic labeling method. In said marking system, different label reagents can be used in one labeling method. For instance, different fluorescence reagents with different wavelength are bound to different ligands so as to form distinctive markers.

In this invention, the “ligand” refers to one molecule presenting affinity to the target molecule. For example, in some markers used in EXAMPLES (e.g. fluorescence-labeled antigen, fluorescence-labeled antibody or fluorescence-labeled anti-antibody), the antibody, the antigen or the anti-antibody is the ligand and the fluorescence reagent is the label reagent. Selection of the ligands, used for the markers of the subject kit, depends to the probe combination selected and the lignd specific reactivity, etc.

The lignd used in the kit of this invention presents antibody-antigen reactivity or antigen or/and antibody affinity. In this invention, the antigen or/and antibody affinity of the ligand is a reactivity based on affinity mechanisms rather than antigen-antibody reaction mechanism, such as the respective affinity of biotin, avidin, staphylococcus A (SPA), (staphylococcus G) SPG, psoralen, digoxin with antigen and/or antibody.

In this invention, the “antigen-antibody reactivity” between an antigen and an antibody refers not only to the specific reactivity between an antigen and its pair antibody, but also to the species-specific reactivity and the structure-specific reactivity; the “specific reactivity” refers to an antigen-antibody reactivity between an antigen and its pair antibody; the “species-specific reactivity” refers to the antigen-antibody reactivity based on different reactivities of an antibody with different anti-antibodies prepared from different species of animals; and the “structure-specific reactivity” refers to the antigen-antibody reactivity based on different reactivities of a type or subtype of immunoglobulin with different anti-antibodies prepared by using different types or subtypes of immunoglobulin or their characteristic segments. One example for the specific reactivity is that between human HCV antigen and its pair human HCV antibody. One example for the species-specific reactivity is that a goat anti-antibody against human antibody reacts specifically with a human antibody against HBsAg but does not react significantly with a mouse mAb against HBsAg. One example for the structure-specific reactivity is that a human anti-HBV IgG has a significant reaction with an Anti-antibody against human IgG but not with an anti-antibody against human IgM.

The biochip kit of this invention is characterized also by its marker combination. The markers in the kit are to be one of the following marker combinations:
A. the combination of labeled specific antibody and labeled specific antigen;
B. the combination of labeled anti-antibodies with different structure-specificities;
C. the combination of labeled anti-antibody with species-specificity and labeled specific antibody and/or labeled specific antigen;
D. the combination of labeled anti-antibody with structure-specificity and labeled specific antibody and/or labeled specific antigen;
E. any combination derived from the combinations mentioned-above.

In this invention, the markers used in the subject kit is not simple labeled ligand, such as one or several antibodies or labeled antigens, or a labeled anti-antibody, but a maker combination, such as the combination of labeled specific antibody and labeled specific antigen, the combination of labeled anti-antibody with species-specificity and labeled specific antibody, the combination of several labeled anti-antibodies with different species-specificities, etc. Selection of the marker combination (the sort and the number of the ligands labeled) depends on the following factors: the probe combination selected; the detection method combination selected; and the specific reactivities of the ligands. An example for the combination A is the following combination: Rhodamine-labeled specific antigens (e.g. HCV, HIV, HTLV, syphilis specific antigens) and a Rhodamine-labeled specific antibody (e.g. an antibody against HBs). An example for the combination B is the following combination: a Rhodamine-labeled anti-antibody against IgM and a Rhodamine-labeled anti-antibody against IgG. An example for the combination C is the following combination: a Rhodamine-labeled goat anti-human and a Rhodamine-labeled mouse mAb against HBsAg. An example for the combination D is the following combination: a Rhodamine-labeled anti-antibody against IgM and a Rhodamine-labeled HBsAg.

In this invention, the markers in the marker combination can be added to the reactor indifferent patterns (respective addition, partially or wholly mixed addition).

The biochip kit of this invention is characterized also by the probe antibody or/and the probe antigen immobilized in a reactor.

In the kit, said probe antibody includes antibody against HBsAg (HbsAb), such as the kits of the EXAMPLES 1, 2, 6, 7 and 8. And said probe antigen includes human immuno-deficient virus (HIV) antigen, hepatitis C virus (HCV) antigen and syphilis antigen, such as the kits of the EXAMPLES 1 and 2; or said probe antigen includes hepatitis B core antigen (HBcAg), hepatitis B surface antigen (HbsAg) and hepatitis B e antigen (HBeAg), and said probe antibody includes also hepatitis B e antibody (HBeAb), such as the kit of the EXAMPLE 6; or said probe antigen includes hepatitis C virus antigen (HCV-Ag) and hepatitis G virus antigen (HGV-Ag), such as the kit of the EXAMPLE 8.

In the kit, said probe antibody includes antibody against anti-HIV p24 antigen, and said probe antigen includes HIV gp160, HIV gp41and HIV gp36 epitopes, such as the kits of the EXAMPLES 3 and 4.

In the kit, said probe antigen includes Epstein-Barr virus (EBV) antigen (EBV-Ag), such as the kit of the EXAMPLE 5. In the kit, said probe antibody includes: antibody against human chorionic gonadotrophin (HCG), anti-tumor marker antigen 50 (CA50) antibody, anti-saccharide antigen 242 (CA242) antibody, anti-mammary cancer specific antigen (CA153) antibody, anti-carcinoembryonic antigen (CEA) antibody, and anti-oophoroma specific antigen (CA125) antibody.

The kits of this invention can be used to detect one of the following 6 different target molecule combinations:
A. the combination of antigen and antibody;
B. the combination of different types of immunoglobulins;
C. the combination of different subtypes of immunoglobulins;
D. the combination of antigen and different types of immunoglobulin and /or the mixture of different types of immunoglobulin;
E. the combination of antigen and different subtypes of immunoglobulin and /or the mixture of different types of immunoglobulin;
F. any derived combination thereof.

In another embodiment, this invention is also directed to a method for biological sample analysis with biochip, comprising:
(1) subjecting the biological sample to the reactor in the biochip of the subject biochip kit, and making them react;
(2) optionally, subjecting marking reagents to said reactor in the form of individual substance, partial mixture or complete mixture, and
(3) analyzing results in the reactor after the reaction.

The advantage of the kit and the method of this invention is: one reactor can be used to test different target molecules such as antibody and antigen, different types of immunoglobulins, different subtypes of immunoglobulins etc. Thereby the number of reactors and time needed for testing the combination are decreased, while the assay comparability is improved. The kit in this invention is economical, timesaving and efficient.

Followings are the detailed examples of this invention. All of the substrates for preparing the biochip of this invention are available in domestic mark, or made and provided by SEDAC Corp. France.

### EXAMPLE 1

### A Biochip Screening Kit for Blood Transfusion (1)

In this example, the kit consists of screening biochip for blood transfusion, markers, negative control, positive control, buffers for washing, etc. The kit is to be used for detecting Hepatitis B surface antigen (HbsAg), Hepatitis C virus (HCV) antibodies, HIV₁₊₂ antibodies and syphilis antibodies in human serum.

The probe combination used in the subject kit, designed in accordance with the blood screening items required by law, is that of one antibody and 3 antigens. The probe antibody selected is a rabbit antibody against human HBsAg. The probe antigens selected are respectively a multiple-epitope-fusion HCV antigen, a multiple-epitope-mixing HIV₁₊₂ antigen and a multiple-epitope-mixing syphilis antigen. The probes at its optimized concentration are respectively spotted on a substrate in the form of a 4×3 micro-array (each probe spotted in 3 spots), and then bovine albumin is used to inactivate residual surface of the substrate. The biochip is then dried and ready for use.

The detection method used for the subject kit is a combination method of the indirect detecting method and the bi-antibody sandwich method. The marker combination used in the subject kit is that of one labeled specific antibody and one labeled species-specific anti-antibody. The ligand antibody in the labeled specific antibody is a specific mouse mAb against HBsAg, which presents the specific reactivity with its pair target antigen (the human HbsAg) captured by the probe antibody but is not reactive with the probe antigens, thereby a bi-antibody sandwich (the probe antibody/the target antigen/the ligand antibody) is formed. The ligand anti-antibody in the labeled species-specific anti-antibody is a goat anti-antibody against human antibodies, which reacts with the target human antibodies (human HCV Ab, HIV₁₊₂ Ab and syphilis Ab) captured respectively by the probe antigens, but is not reactive with the probe rabbit antibody (the rabbit antibody against human HBsAg) and the ligand mouse antibody (the mouse mAb against HBsAg), according to the species-specific reactivity. In this example, rhodamine is used as the label reagent and is bound to the ligands. The ligands are labeled with the rhodamine under optimized conditions, respectively. The markers prepared are ready for use in the form of a mixture or of two separated markers.

The negative control of this chip kit is a human serum negative in HBsAg, HCV antibody, HIV₁₊₂ antibody and syphilis antibody, selected by ELISA kits. The ELISA probe-plates used in the ELISA kits are micro-well plates coated respectively with rabbit antibody against HBsAg, multiple-epitope-fusion HCV antigen, multiple-epitope-mixing HIV₁₊₂ antigen or syphilis antigen. The positive control of this chip kit is a mixture of HBsAg positive sera, HCV antibody positive sera, HIV₁₊₂ antibody positive sera and syphilis antibody positive sera, selected by using the ELISA kits.

This biochip screening kit for blood transfusion can be used for detecting the possible presence of HBsAg, HCV Ab, HIV₁₊₂ Ab and syphilis Ab in a human serum sample.

The human serum samples in this experiment are: 1. HbsAg positive human serum, 2.HCV antibody positive human serum, 3.HIV₁₊₂ antibody positive human serum, 4. Syphilis antibody positive human serum, 5.HbsAg positive and syphilis positive human serum, 6.Negative control and7. Positive control. These human serum samples are previously detected using the ELISA kits.

The samples are detected as following: one of the human serum samples is diluted and subjected into one reactor of this chip. The antigen-antibody reaction in the reactor is performed at 37° C. for one hour. Then, the unbound serum component is removed from the reactor and the reactor is washed with washing buffer. The marker combination (the mouse mAb against human HBsAg and goat anti-human anti-antibodies labeled with rhodamine) is then subjected into the reactor. The marking reaction is performed at 37° C. for one hour. Then, the reactor is washed first with buffer solution to get rid of unbound markers, and then with anhydrous alcohol. Finally, the dried biochip is scanned and analyzed with a chip scanner (GMS 418 ARRAY SCANNER). The results of the detections with this chip kit are showed in the Table 2.

TABLE 2
Detection results of the biochip screening kit for blood transfusion

| | results using 4 ELISA kits | | | | results using 1 biochip kit | | | |
|---|---|---|---|---|---|---|---|---|
| | | | | syph- | | | | syph- |
| Samples | HbsAg | HCV | HIV | ilis | HbsAg | HCV | HIV | ilis |
| 1 | +* | −* | − | − | + | − | − | − |
| 2 | + | + | − | − | − | + | − | − |
| 3 | − | − | + | − | − | − | + | − |
| 4 | − | − | − | + | − | − | − | + |
| 5 | + | − | − | + | + | − | − | + |
| Negative | − | − | − | − | − | − | − | − |
| control | | | | | | | | |
| Positive | + | + | + | + | + | + | + | + |
| control | | | | | | | | |
| Blank | − | − | − | − | − | − | − | − |

*same as those in Table 1

### EXAMPLE 2

### A Biochip Screening Kit for Blood Transfusion (2)

In this example, the kit consists of the screening biochip for blood transfusion, markers, negative control, positive control, washing solution, etc.

The biochip in this biochip kit is prepared by the same preparation method as that in example 1.

The detection method combination used for the subject kit is that of the bi-antigen sandwich method and the bi-antibody sandwich method. The marker combination used in the subject kit is that of three labeled specific antigens and one labeled specific antibody. The ligand antibody in the labeled specific antibody is a specific mouse mAb against HBsAg, which presents the specific reactivity with its pair target antigen (the human HbsAg) captured by the probe antibody but is not reactive with the probe antigens, thereby a bi-antibody sandwich (the probe antibody/the target antigen/the ligand antibody) is formed. The 3 ligand antigens in the 3 labeled specific antigens are a specific HCV antigen, a specific HIV₁₊₂ antigen and a specific syphilis antigen respectively. Each of the specific ligand antigens presents the specific reactivity with its pair target antibody (the human HBs Ab, the human HIV₁₊₂ Ab or the human syphilis Ab) captured by the corresponding probe antigen but is not reactive with the probe antibody and the ligand antibody, thereby a bi-antigen sandwich (the probe antigen/the target antibody/the ligand antigen) is formed. In this example, rhodamine is used as the label reagent and is bound to the ligands. The ligands are labeled with the rhodamine under optimized conditions, respectively. The markers prepared are ready for use in the form of a mixture or of separated markers.

The negative control and the positive control of this chip kit are the same as those in Example 1.

This biochip screening kit for blood transfusion can be used for detecting the possible presence of HBsAg, HCV antibody, HIV₁₊₂ antibody and syphilis antibody in a human serum sample. The human serum samples in this experiment are the same as those in example 1. The detection of the samples in this example is performed in the same way as in example 1, and the results obtained in the detection are similar to that in table 2 of Example 1.

### EXAMPLE 3

### A Biochip Kit of Detecting HIV Antigen/Antibody (1)

The kit prepared in this example consists of biochip of detecting HIV antigen and HIV antibody, markers, negative controls, positive controls and washing buffer etc. The kit is to be used for detecting the possible presence of HIV antigen and HIV antibody in a human serum sample.

The probe combination in this kit is of that of one antibody and one antigen. The probe antibody selected is a mouse mAb against HIV p24; and the probe antigen selected is a mouse multiple-epitope-mixing antigen comprising HIV gp160, gp41, gp36. The probes at its optimized concentration are respectively spotted on a substrate in the form of a 2×3 micro-array (each probe spotted in 3 spots), and then bovine albumin is used to block residual surface of the substrate. The biochip is then dried and ready for use.

The detection method combination used for the subject kit is that of the bi-antigen sandwich method and the bi-antibody sandwich method. The marker combination used in the subject kit is that of one labeled specific antibody and one labeled specific antigen. The ligand antibody in the labeled specific antibody is a specific mouse mAb against HIV p24, which presents the specific reactivity with its pair target antigen (the human HIV Ag ) captured by the probe antibody but is not reactive with the probe antigen, thereby a bi-antibody sandwich (the probe antibody/the target antigen/the ligand antibody) is formed. The ligand antigen in the labeled specific antigen is a specific HIV₁₊₂ antigen, which presents the specific reactivity with its pair target antibody (the human HIV₁₊₂ Ab) captured by the probe antigen but is not reactive with the probe antibody and the ligand antibody, thereby a bi-antigen sandwich (the probe antigen/the target antibody/the ligand antigen) is formed. In this example, rhodamine is used as the label reagent and is bound to the ligands. The ligands are labeled with the rhodamine under optimized conditions, respectively. The markers prepared are ready for use in the form of a mixture or of separated markers.

The negative control in this chip kit is a HIV antibody and antigen negative human serum, selected by using ELISA kits. The ELISA probe-plates used in the ELISA kits are micro-well plates coated respectively with the anti-HIV p24 mAb and the mouse multiple-epitope-mixing antigen comprising HIV gp160, gp41, gp36. The positive control in this chip kit is a HIV antibody and antigen positive human serum, selected by using the ELISA kits.

This biochip kit can be used for testing the possible presence of HIV antibody and HIV antigen in a human serum sample. The human serum samples in this experiment are: A. HIV p24 antigen positive human serum, B. HIV gp160, gp41, gp36 antibody positive human serum, C. HIV antigen and antibody positive human serum, D. negative control, E. positive control. These human serum samples are previously detected by using the ELISA kits mentioned above.

The samples are detected as following: one of the human serum samples is diluted and subjected into one reactor of this chip. The antigen-antibody reaction in the reactor is performed at 37° C. for one hour. Then, the unbound serum component is removed from the reactor and the reactor is washed with washing buffer. The marker combination is then subjected into the reactor. The marking reaction is performed at 37° C. for one hour. Then, the reactor is washed first with buffer solution to get rid of unbound markers, and then with anhydrous alcohol. Finally, the dried biochip is scanned and analyzed with a chip scanner (GMS 418 ARRAY SCANNER). The results of the detections with this chip kit are showed in the Table 3.

TABLE 3
The results of HIV antigen/antibodies detecting
biochip kit

| | |
|---|---|
| results using 2 ELISA | results using the |
| kits | biochip kit |

| | Ab coated | Ag coated | Probe Ab | Probe Ag |
|---|---|---|---|---|
| Sample | well | well | spot | spot |
| A | +* | −* | + | − |
| B | − | + | − | + |
| C | + | + | + | + |
| Negative | − | − | − | − |
| control | | | | |
| Positive | ++++ | ++++ | ++++ | ++++ |
| control | | | | |
| Blank | − | − | − | − |

*same as those in Table 1

### EXAMPLE 4

### A Biochip Kit of Detecting HIV Antigen/Antibody (2)

The kit prepared in this example consists of biochip of detecting HIV antigen and HIV antibody, markers, negative controls, positive controls and washing buffer etc. The kit is to be used for detecting the possible presence of HIV antigen and HIV antibody in human serum sample.

The biochip in this biochip kit is prepared by the same preparation method as that in example 1.

The detection method used for the subject kit is a combination method of the indirect detecting method and the bi-antibody sandwich method. The marker combination used in the subject kit is that of one labeled specific antibody and one labeled species-specific anti-antibody. The ligand antibody in the labeled specific antibody is a specific mouse mAb against HIV p24, which presents the specific reactivity with its pair target antigen (the human HIV Ag) captured by the probe antibody but is not reactive with the probe antigen, thereby a bi-antibody sandwich (the probe antibody/the target antigen/the ligand antibody) is formed. The ligand anti-antibody in the labeled species-specific anti-antibody is a goat anti-antibody against human antibodies, which reacts with the target human antibodies (human HIV₁₊₂ Ab) captured by the probe antigen, but is not reactive with the probe mouse antibody and the ligand mouse antibody, according to the species-specific reactivity. In this example, rhodamine is used as the label reagent and is bound to the ligands. The ligands are labeled with the rhodamine under optimized conditions, respectively. The markers prepared are ready for use in the form of a mixture or of separated markers.

The negative control and positive control of this chip kit are the same as those in example 3.

The human serum samples in this experiment are the same as those in example 3. The detection of the samples is performed in this example same as that in example 1, and the results obtained in the detection are similar to that in table 3 of example 3.

### EXAMPLE 5

### A Biochip Kit for Testing Antigen/Antibody Related to Tumors

The biochip kit prepared in this example consists of biochip, labeled material, negative control, positive control, reference materials, washing solution and etc. The biochip kit in this example is to be used for detecting the possible presence of antigens and antibodies related to tumors in human serum samples.

The probe combination in biochip in this biochip kit is determined depending on the detection requirement and the available tumor related antigens/antibodies. In this example, the probe combination is antibody and antigen combination. The antigen probe in the probe combination is EBV antigen of synthetic polypeptide. The antibody probes in the probe combination are six mouse antibodies respectively against HCG trophic hormone, tumor marker antigen 50 (CA50), saccharide antigen 242 (CA242), mammary cancer specific antigens (CA153), carcinoembryonic antigen (CEA) and oophoroma specific antigen (CA125). The antigen and antibody probes at optimal concentration are respectively immobilized on the activated substrate in a form of 7×3 array (3 spots for each type of probe) by using a manual spotter. After spotting, bovine albumins is used to block the activated surface of the matrix. Then the biochip is dried and ready for use.

The detection method used for the subject kit is a combination method of the indirect detecting method and the bi-antibody sandwich method. The marker combination used in the subject kit is that of six labeled specific antibodies and one labeled species-specific anti-antibody. The 6 ligand antibodies in the labeled specific antibodies are a specific mouse mAb against HCG, a specific mouse mAb against CA50, a specific mouse mAb against CA242, a specific mouse mAb against CA153, a specific mouse mAb against CEA, and a specific mouse mAb against CA125), respectively. Each of these 6 specific ligand antibodies presents the specific reactivity with its pair target antigen (the human HCG Ag, CA50 Ag, CA242 Ag, CA153 Ag, CEA Ag, or CA125HIV Ag) captured by the corresponding probe antibody but is not reactive with the probe antigen, thereby a bi-antibody sandwich (the probe antibody/the target antigen/the ligand antibody) is formed. The ligand anti-antibody in the labeled species-specific anti-antibody is a goat anti-antibody against human antibodies, which reacts with the target human antibody (human EBV VCA Ab) captured by the probe antigen, but is not reactive with the 6 probe mouse antibodies and the 6 ligand mouse antibodies, according to the species-specific reactivity. In this example, rhodamine is used as the label reagent and is paird on the ligands. The rhodamination of the ligands is realized respectively under optimized conditions. And then, the makers can be used in a mixed or separated form.

The negative control in this kit is the negative human serum selected through 7 ELISA kits, each of which includes a microwell probe-plate coated with one of the following probes: synthetic polypeptides EBV VCA antigen, antibody against HCG, antibody against tumor marker antigen 50 (CA50), the antibodies against polysaccharide and glucoprotein, such as saccharide antigen 242 (CA242), antibody against mammary cancer specific antigen (CA153), antibody against carcinoembryonic antigen (CEA) and antibody against oophoroma specific antigen (CA125). The quality controls in this kit are the tumor references selected through other quantitative test.

By using this biochip kit, one antibody and six antigens related to the tumors in one sample can be quantitatively detected in one reactor. So the kit has advantage in saving time and money.

### EXAMPLE 6

### A Biochip Kit of Detecting HBV Antibody/Antigen

The biochip kit prepared in this example consists of biochip of detecting antibody and antigen related to hepatitis B virus (HBV), markers, negative control, positive control, washing solution and etc. This kit is for the detection of hepatitis B virus (HBV) antigens/antibodies in human serum sample.

The probe combination used in the subject kit, designed according to the detection objective and the available HBV related antigens/antibodies, includes two antibodies and 3 antigens. The two probe antibodies, selected for the probe combination, are an isolated mouse mAb against human HBsAg (hepatitis B surface antigen) and an isolated mouse mAb against human HBeAb (hepatitis B e antigen), respectively. The three probe antigens, selected for the probe combination, are an isolated HBsAg (hepatitis B surface antigen), an isolated HBcAg (hepatitis B core antigen) and a isolated HBeAg (hepatitis B e antigen). The probes at its optimized concentration are respectively spotted on a substrate in the form of a 5×3 micro-array (each probe spotted in 3 spots), and then bovine albumin is used to inactivate residual surface of the substrate. The biochip is then dried and ready for use.

The kit prepared in this example is to be used for the detection of hepatitis B surface antigen, hepatitis B e antigen, hepatitis B surface antibodies IgG, hepatitis B surface antibodies IgM, hepatitis B e antibodies IgG, hepatitis B e antibodies IgM, hepatitis B c antibodies IgG, hepatitis B c antibodies IgM in a human serum sample.

The detection method used for the subject kit is a combination method of the indirect detecting method and the bi-antibody sandwich method. The marker combination used in the subject kit is that of two labeled specific antibodies and two labeled species-specific and structure-specific anti-antibodies. The 2 ligand antibodies in the 2 labeled specific antibodies are a specific mouse mAb against HbsAg and a specific mouse mAb against HbeAg, respectively. Each of the 2 ligand antibodies presents the specific reactivity with its pair target antigen (the human HbsAg or the human HbeAg) captured by the corresponding probe antibody, and is not reactive with another target antigen and the probe antigen epitopes, thereby a bi-antibody sandwich (the probe antibody/the target antigen/the ligand antibody) is formed. The 2 ligand anti-antibodies in the 2 labeled species-specific and structure-specific anti-antibodies are a goat anti-antibody against human IgG and a goat anti-antibody against human IgM. Each of the anti-antibodies presents the species-specific reactivity and the structure reactivity with its pair target human antibody (human IgG or IgM) captured by the corresponding probe antigens, but is not reactive with the probe mouse antibodies, the ligand mouse antibodies, and other type of human antibody, according to the species-specific reactivity and the structure reactivity. In this example, rhodamine (excitation wavelength λ ex=546 nm and emission wavelength λ em=575 nm) is used as the label reagent for labeling the two specific antibodies and the goat anti-human IgG. However, Cy5 (excitation wavelength λ ex=649 nm and emission wavelength λ em=670 nm) is used as the label reagent for labeling the goat anti-human IgM. The ligands are labeled with the rhodamine and Cy5 under optimized conditions, respectively.

The negative control in this kit is the negative human serum selected through ELISA method. The positive control in this example is the mixture of positive human serum selected through ELISA detection. The positive samples in this experiment respectively are: 1#. hepatitis B surface antigen positive sample, 2#. hepatitis B e antigen positive sample, 3#. hepatitis B surface antibodies IgG positive sample, 4#. hepatitis B surface antibodies IgM positive sample, 5#. hepatitis B e antibodies IgG positive sample, 6#. hepatitis B e antibodies IgM positive sample, 7#. hepatitis B c antibodies IgG positive sample, 8#. hepatitis B c antibodies IgM positive sample. The negative and positive results of all these selected human serum samples are pre-examined through ELISA.

The samples are detected as following: one of the human serum samples is diluted and subjected into one reactor of this chip. The antigen-antibody reaction in the reactor is performed at 37° C. for one hour. Then, the unbound serum component is removed from the reactor and the reactor is washed with washing buffer. The marker combination (the mouse antibody against HBsAg labeled with rhodamine, the mouse antibody against HBeAg labeled with rhodamine, the goat anti-human IgG labeled with rhodamine, an the goat anti-human IgM labeled with Cy5) is then subjected into the reactor. The marking reaction is performed at 37° C. for one hour. Then, the reactor is washed first with buffer solution to get rid of unbound markers, and then with anhydrous alcohol. Finally, the dried biochip is scanned and analyzed with a multi-wavelength laser confocal microscopy (GMS 418 ARRAY SCANNER). The excitation wavelength of 540 nm is used to obtain the detection results of hepatitis B surface antigen, hepatitis B e antigen, hepatitis B surface antibodies IgG, hepatitis B e antibodies IgG, hepatitis B c antibodies IgG. And the excitation wavelength of 650 nm is used to detect hepatitis B surface antibody IgM, hepatitis B e antibody IgM, hepatitis B c antibody IgM. (Table 4):

TABLE 4
The results of biochip kit for the detection of
hepatitis B in human serum samples

| | Results using 8 ELISA kits | | | | | | | | Results using one biochip kit | | | | | | | |
|---|---|---|---|---|---|---|---|---|---|---|---|---|---|---|---|---|
| Sample | A | B | C | D | E | F | G | H | A | B | C | D | E | F | G | H |
| 1# | +* | −* | − | − | − | − | − | − | + | − | − | − | − | − | − | − |
| 2# | − | + | − | − | − | − | − | − | − | + | − | − | − | − | − | − |
| 3# | − | − | + | − | − | − | − | − | − | − | + | − | − | − | − | − |
| 4# | − | − | − | + | − | − | − | − | − | − | − | + | − | − | − | − |
| 5# | − | − | − | − | + | − | − | − | − | − | − | − | + | − | − | − |
| 6# | − | − | − | − | − | + | − | − | − | − | − | − | − | + | − | − |
| 7# | − | − | − | − | − | − | + | − | − | − | − | − | − | − | + | − |
| 8# | − | − | − | − | − | − | − | + | − | − | − | − | − | − | − | + |
| Negative | − | − | − | − | − | − | − | − | − | − | − | − | − | − | − | − |
| control | | | | | | | | | | | | | | | | |
| Positive | + | + | + | + | + | + | + | + | + | + | + | + | + | + | + | + |
| control | | | | | | | | | | | | | | | | |
| Blank | − | − | − | − | − | − | − | − | − | − | − | − | − | − | − | − |

*same as those in Table 1
Notes:
A: Hbs Ag;
B: HBe Ag;
C: Hbs IgG;
D: Hbs IgM;
E: Hbe IgG;
F: Hbe IgM;
G: Hbc IgG;
H: HBc IgM

### EXAMPLE 7

### A Biochip Kit of Detecting HBV and HCV Antigen/Antibody

This kit is for detecting antigen/antibody of hepatitis B virus (HBV) and hepatitis C virus (HCV) in human serum sample.

In this example, the kit consists of biochip, markers, negative control, positive control, washing solution and etc.

Selection of the probe combination in the biochip depends on the requirement of detection and the available related antigen/antibody. In this example, the antibody/antigen combination is selected as the probe combination. The antibody probe in the probe combination is rabbit antibody against HBsAg, and the antigen probe in the probe combination is hepatitis C fusion antigen. The antibody probe is used for detecting hepatitis B surface antigen and the antigen probe for detecting hepatitis C antibody in a human serum sample. The antigen and antibody probes at optimal concentration are respectively immobilized on the activated substrate in a form of 2×3 array (3 spots for each type of probe) by using a manual spotter. Then, bovine albumin is used to block the surface of the matrix. The biochip is then dried and ready for use.

The detection method used for the subject kit is a combination method of the indirect detecting method and the bi-antibody sandwich method. The marker combination used in the subject kit is that of one labeled specific antibody and one labeled species-specific anti-antibody. The ligand antibody in the labeled specific antibody is a specific mouse mAb against HBsAg, which presents the specific reactivity with its pair target antigen (the human HbsAg) captured by the probe antibody but is not reactive with the probe antigens, thereby a bi-antibody sandwich (the probe antibody/the target antigen/the ligand antibody) is formed. The ligand anti-antibody in the labeled species-specific anti-antibody is a goat anti-antibody against human antibodies, which reacts with the target human antibodies (human HCV Ab) captured by the probe antigen, but is not reactive with the probe rabbit antibody (the rabbit antibody against human HBsAg) and the ligand mouse antibody (the mouse mAb against HBsAg), according to the species-specific reactivity. In this example, rhodamine is used as the label reagent and is bound to the ligands. The ligands are labeled with the rhodamine under optimized conditions, respectively. The markers prepared are ready for use in the form of a mixture or of two separated markers.

The other accessory parts of the kit prepared in this example are negative control, positive control and washing solution, etc.

### EXAMPLE 8

### A Biochip Kit of Detecting Hepatitis Virus Antigen/Antibody

This kit prepared in the Example consists of biochip, markers, negative control, positive control, washing solution and etc. This kit is for detecting antibody and antigen related to different hepatitis virus.

The probe combination used in the subject kit, designed according to the detection objective and the available significative antigen and antibody related to different hepatitis virus, includes 1 antibody and 5 antigens. The probe antibody, selected for the probe combination, is an isolated rabbit mAb against human HBsAg (hepatitis B surface antigen). The five probe antigens, selected for the probe combination, are a HAV Ag (hepatitis A antigen), a multiple-epitope-fusion HCV Ag (hepatitis C virus antigen), a multiple-epitope-fusion HDV Ag (hepatitis D virus antigen), a multiple-epitope-fusion HGV Ag (hepatitis G virus antigen), and a multiple-epitope-fusion HEV Ag (hepatitis E virus antigen). The probes at its optimized concentration are respectively spotted on a substrate in the form of a 6×3 micro-array (each probe spotted in 3 spots), and then bovine albumin is used to inactivate residual surface of the substrate. The biochip is then dried and ready for use.

The kit prepared in this example is to be used for the detection of HAV antibody, HBs antigen, HCV antibody, HDV antibody and HEV antibody in a human serum sample.

The detection method used for the subject kit is a combination method of the indirect detecting method and the bi-antibody sandwich method. The marker combination used in the subject kit is that of one labeled specific antibody and one labeled species-specific anti-antibody. The ligand antibody in the labeled specific antibody is a specific mouse mAb against HBsAg, which presents the specific reactivity with its pair target antigen (the human HBsAg) captured by the probe antibody but is not reactive with the probe antigens, thereby a bi-antibody sandwich (the probe antibody/the target antigen/the ligand antibody) is formed. The ligand anti-antibody in the labeled species-specific anti-antibody is a goat anti-antibody against human antibodies, which reacts with the target human antibodies (human HAV Ab, HCV Ab, HDV Ab, HGV Ab or HEV Ab) captured respectively by the probe antigens, but is not reactive with the probe rabbit antibody (the rabbit antibody against human HBsAg) and the ligand mouse antibody (the mouse mAb against HBsAg), according to the species-specific reactivity. In this example, rhodamine (excitation wavelength λ ex=546 nm and emission wavelength λ em=575 nm) is used as the label reagent and is bound to the ligands. The ligands are labeled with the rhodamine under optimized conditions, respectively. The markers prepared are ready for use in the form of a mixture or of two separated markers.

Other accessory parts of the kit prepared in this example are negative control, positive control and washing solution, etc.

1. A biochip kit, comprising at least one biochip with at least one reactor, in which at least probe antibody and probe antigen are immobilized non-randomly in the form of probes array or/and probes pattern.

2. The kit of claim 1, comprising also markers, wherein: said markers consist of ligands, corresponding to said probe antibody and probe antigen, and label reagent bound to the ligands; and said ligand presents specific antibody-antigen reactivity, or antigen affinity or/and antibody affinity.

3. The kit of claim 2, wherein said markers are one of the following marker combinations:
A. the combination of labeled specific antibody and labeled specific antigen;
B. the combination of labeled anti-antibody with different structure-specificity ;
C. the combination of labeled anti-antibody with species-specificity and labeled specific antibody and/or labeled specific antigen;
D. the combination of labeled anti-antibody with structure-specificity and labeled specific antibody and/or labeled specific antigen;
E. any combination derived from the combinations mentioned above.

4. The kit of claim 1, wherein said probe antibody includes antibody against hepatitis B surface antigen.

5. The kit of claim 4, a screening kit, wherein said probe antigen includes human immuno-deficient virus antigen, hepatitis C virus antigen and syphilis antigen.

6. The kit of claim 4, a kit of detecting hepatitis B virus antigen/antibody, wherein:
said probe antibody includes also hepatitis B e antibody; and
said probe antigen includes hepatitis B core antigen, hepatitis B surface antigen and hepatitis B e antigen.

7. The kit of claim 4, a kit of detecting hepatitis virus antigen/antibody, wherein said probe antigen includes hepatitis C virus antigen and hepatitis G virus antigen.

8. The kit of claim 1, a kit of detecting HIV antigen/antibody, wherein:
said probe antibody includes antibody against anti-HIV p24 antigen;
said probe antigen includes HIV gp160, HIV gp41 and HIV gp36.

9. The kit of claim 1 or claim 3, a kit of testing antigen/antibody related to tumors, wherein said probe antigen includes Epstein-Barr virus (EBV) antigen (EBV- Ag).

10. The kit of claim 9, wherein said probe antibody includes: antibody against human chorionic gonadotrophin(HCG), anti-tumor marker antigen 50 (CA50) antibody, anti-saccharide antigen 242 (CA242) antibody, anti-mammary cancer specific antigen (CA153) antibody, anti-carcinoembryonic antigen (CEA) antibody, and anti-oophoroma specific antigen (CA125) antibody;

11. A method for biological sample analysis with biochip, comprising:
(1) subjecting the biological sample to the reactor in the biochip of claim 1, and making them react;
(2) optionally, subjecting marking reagents to said reactor in the form of individual substance, partial mixture or complete mixture, and
(3) analyzing results in the reactor after the reaction.
